(19) United States
(12) Patent Application Publication
(10) Pub. No.: US 2021/0324319 A1
(43) Pub. Date: Oct. 21, 2021

(54) CELL STRUCTURE MANUFACTURING SYSTEM

(71) Applicant: CYFUSE BIOMEDICAL K.K., Tokyo (JP)

(72) Inventors: Yasuto KISHII, Tokyo (JP); Norihiko TOKUNAGA, Tokyo (JP)

(73) Assignee: CYFUSE BIOMEDICAL K.K., Tokyo (JP)

(21) Appl. No.: 16/616,284

(22) PCT Filed: Feb. 1, 2019
(86) PCT No.: PCT/JP2019/003743

§ 371 Date: Nov. 22, 2019

(51) Int. Cl. C12M 1/26; C12M 3/00; C12M 1/32; C12M 1/00

(52) U.S. Cl. CPC C12M 33/00; C12M 23/48; C12M 23/42; C12M 23/50; C12M 23/12

(57) ABSTRACT

The cell structure manufacturing system including a cabinet being a closed space capable of maintaining an inside at a high cleanness, a cultivation plate supplying device configured to supply one cultivation plate of a plurality of cultivation plates to a conveying device in a cultivation plate supplying area, a transfer device including a cell aggregate injection device configured to discharge and transfer, and a sticking device including a needle-like body clamping mechanism configured to remove one needle-like body from a needle-like body supplying mechanism storing a plurality of needle-like bodies and perform gripping of the one needle-like body.

### TECHNICAL FIELD

The present invention relates to a cell structure manufacturing system for manufacturing the three-dimensional structure of cells.

### BACKGROUND ART

Conventionally, as disclosed in PTL 1 and PTL 2, the technique has been disclosed that utilizes the character of cell aggregates that the contacting cell aggregates are fused, skewers the cell aggregates by needle-like bodies so that the cell aggregates contact to each other, and cultivates and matures the cell aggregates for a predetermined period in the state where the cell aggregates skewered by the respective needle-like bodies contact to each other even between the adjacent needle-like bodies, so as to form a cell structure. Within the period for cultivating the cell aggregates, the needle-like bodies play a role of a provisional base material that supports the cell aggregates. Here, a cell aggregate refers to the cell aggregate formed as a lump of a plurality of cells by combined cultivation of a single cell, for example, spheroid.

In these techniques, first, a plurality of single cells are cultivated in a cell aggregate cultivation plate (hereinafter, “the cultivation plate”), and a plurality of cell aggregates are formed. As shown in FIG. 9A, in a cultivation plate 81, a culture solution is filled in a cell aggregate receiving hole (hereinafter, the well) 81c for each cell aggregate S in the cultivation plate 81, and the cell aggregate S generated from cells is immersed. Therefore, the well 81c without a hole in the bottom is used, so that the culture solution can be well maintained for a cultivation period. Here, a first technique disclosed in PTL 1 is the technique that sucks and removes the cell aggregate S one by one from the well 81c of the cultivation plate 81, and sticks the sucked cell aggregate S to the fixed needle-like body while maintaining and moving the sucked cell aggregate S. Accordingly, in this first technique, the cultivation plate 81 including the well 81c without a hole in the bottom in the stage of forming a cell aggregate can be used as it is.

On the other hand, a second technique disclosed in PTL 2 is a technique that moves the needle-like body toward a cell aggregate, without moving the cell aggregate. That is, in the second technique, the needle-like body is repeatedly moved up and down to cell aggregates arranged not to move, so as to produce a plurality of needle-like bodies with a plurality of cell aggregates skewered to be adjacent to each other. Then, these needle-like bodies are aligned in an alignment frame, and cultivated and matured only for a predetermined period, so as to form a cell structure. In the second technique, even if it is attempted to stick the needle-like body to the cell aggregate S while being received by the well 81c without a hole in the bottom, the tip of the needle-like body collides with the bottom of the well 81c, and it is impossible to penetrate through the cell aggregate.

Therefore, in the second technique, with an aim to stick the cell aggregates S so as to penetrate through the cell aggregates S in order to make the needle-like body with the skewered cell aggregates S, it is necessary to transfer the cell aggregate S to a stacking tray 83 that includes a through-hole 82b through which a needle-like body can penetrate at the bottom as shown in FIG. 9B, or includes a cell aggregate holding hole (hereinafter, the well hole) 82a for maintaining the cell aggregate, and including at the bottom a porous material 82c through which the needle-like body can penetrate as shown in FIG. 9C.

Then, after transferring the cell aggregate S to the stacking tray 83 with the well hole 82a enabling a needle-like body to penetrate through the bottom, a thin needle-like body is repeatedly moved up and down to a plurality of cell aggregates, and the needle-like body with a plurality of adjacent skewered cell aggregates is made. By repeating this, a plurality of needle-like bodies with a plurality of adjacent skewered cell aggregates are made. A cell structure is formed by fixing these plurality of needle-like bodies with a plurality of adjacent skewered cell aggregates in a predetermined shape, and cultivating and maturing the plurality of needle-like bodies with a plurality of adjacent skewered cell aggregates only for predetermined period. The matured and formed cell structure is removed from the needle-like bodies, and is used as, for example, a cell product for a regeneration medicine application or a drug development application.

### CITATION LIST

### Patent Literature

PTL 1: Specification of Japanese Patent No. 4517125
PTL 2: Pamphlet of International Publication No. WO2016/047737

### SUMMARY OF INVENTION

### Technical Problem

Since a subject is a cell in the formation of a cell structure, it is vulnerable to an environmental variation, bacteria, etc. of the external world. Accordingly, it is preferable to perform a process of transferring cell aggregates to a stacking tray to a process of sticking and aligning the cell aggregates as a consistent process in a clean environment that can prevent contamination by maintaining the cleanness at a certain level or more as a compact facility, and can prevent contamination of the cell structure by invasion of an environment outside of the facility, such as especially viruses or bacteria, to the inside of a working space. Additionally, it is also necessary to suppress the infection to an operator who performs access. However, it is difficult to prepare these processes in a large space, and it is necessary to be able to perform these processes in a comparatively local and small space.

### Solution to Problem

A solution is made by a cell structure manufacturing system for forming a cell structure by sticking a cell aggregate by a needle-like body, the cell structure manufacturing system including a cabinet including a closed space whose inside is maintained at a high cleanness, and an access opening enabling access from outside, a cultivation plate supplying device configured to supply one cultivation plate of a plurality of cultivation plates to a conveying device in a cultivation plate supplying area, each cultivation plate having a plurality of cell aggregate receiving holes, the plurality of cell aggregate receiving holes storing externally generated cell aggregates, a transfer device including a cell aggregate injection device configured to discharge and transfer, in a transfer area, the cell aggregates stored in the plurality of cell aggregate receiving holes of the one cultivation plate conveyed by the conveying device respectively to a plurality of cell aggregate holding holes of the stacking tray having the plurality of cell aggregate holding holes each allowing penetration of a bottom by the needle-like body, and a sticking device including a needle-like body clamping mechanism configured to remove one needle-like body from a needle-like body supplying mechanism storing a plurality of needle-like bodies and perform gripping of the one needle-like body, the needle-like body clamping mechanism performing continuous sticking of each of the cell aggregates in the plurality of cell aggregate holding holes of the stacking tray by the one needle-like body in a sticking area, the needle-like body clamping mechanism pressing and sticking the needle-like body with the skewered cell aggregates to an alignment base to perform releasing of the gripping.

### Advantageous Effects of Invention

According to the present invention, it is possible to maintain cleanness and prevent contamination, while rendering a process of transferring cell aggregates from a cultivation plate to a stacking tray to a process of sticking and aligning the cell aggregates as a consistent process.

### BRIEF DESCRIPTION OF DRAWINGS

FIG. 1 is a front view showing a cell structure manufacturing system in an embodiment of the present invention.

FIG. 2 is a top view of the inside of the cell structure manufacturing system in the embodiment of the present invention, and shows a cross-section 2-2 of FIG. 1.

FIG. 3 is a function configuration diagram of the cell structure manufacturing system in the embodiment of the present invention.

FIG. 4 is a flowsheet of the cell structure manufacturing system in the embodiment of the present invention.

FIG. 5 is a perspective view showing a cultivation plate supplying device of the cell structure manufacturing system in Example 1 of the present invention.

FIG. 6 is a perspective view showing a transfer device and a sticking device of the cell structure manufacturing system in Example 1 of the present invention.

FIG. 7 shows a flowchart of each process of the cell structure manufacturing system of Example 1 of the present invention.

FIG. 8 is a perspective view showing the sticking device of the cell structure manufacturing system in Example 2 of the present invention.

FIG. 9A is a cross-sectional view of a well portion of the cultivation plate used conventionally and in the embodiment of the present invention.

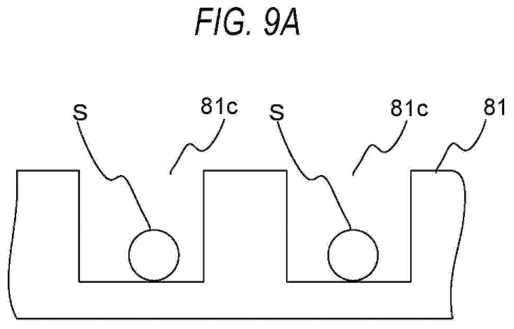

FIG. 9B is a cross-sectional view of an example of a well hole portion of the stacking tray used in the embodiment of the present invention.

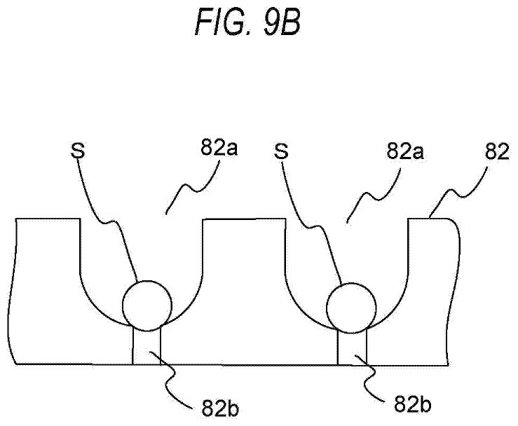

FIG. 9C is a cross-sectional view of another example of the well hole portion of the stacking tray used in the embodiment of the present invention.

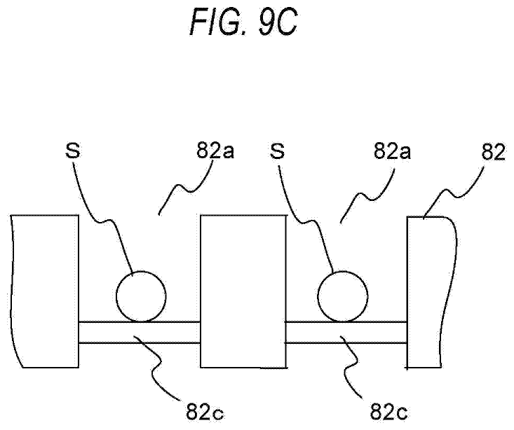

### DESCRIPTION OF EMBODIMENTS

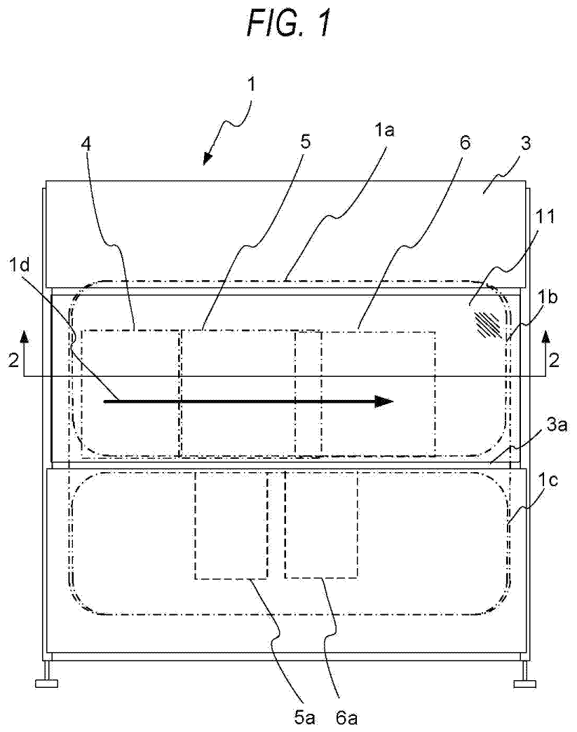

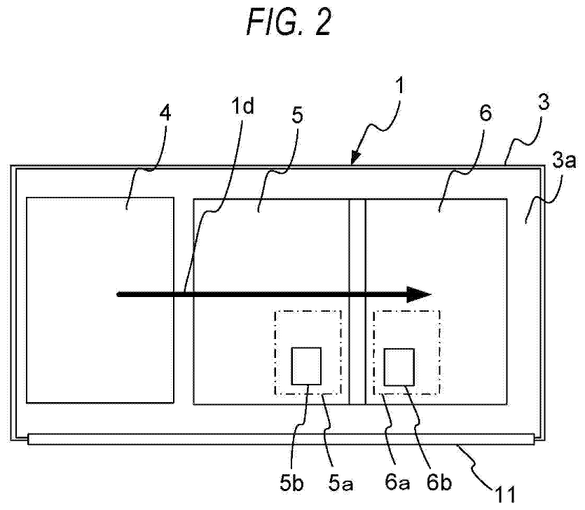

First, referring to FIG. 1 to FIG. 4, a description will be given of a cell structure manufacturing system 1 of an embodiment of the present invention. The cell structure manufacturing system 1 is a system that puts and cultivates a plurality of single cells in a well 81c of a cultivation plate 81 for a predetermined period, and grows the coupled cell aggregates S to form a cell structure. FIG. 1 is a front view of the cell structure manufacturing system 1, and FIG. 2 is a cross-section 2-2 of FIG. 1, and is a top view of the arrangement of an internal space of the cell structure manufacturing system 1. A closed space 1a inside the cell structure manufacturing system 1 is a sealed space that can achieve a clean environment having a predetermined high cleanliness, and typically is a safety cabinet 3. Here, a description will be given on the assumption of the safety cabinet 3. The closed space 1a of the safety cabinet 3 corresponds to a cultivation plate supplying area 4, a transfer area 5, and a sticking area 6. With a focus on a cell aggregate, in the cell structure manufacturing system 1, the cell aggregate is moved from the cultivation plate supplying area 4 to the sticking area 6 via the transfer area 5. The front surface of the safety cabinet 3 serves as an access opening 11 to the cell structure manufacturing system 1, enabling access from the outside. Generally, the access opening 11 is a transparent door that enables observation of the inside of the safety cabinet 3, and that can be opened and closed, and when the access opening 11 is closed, the internal closed space 1a can be maintained in a clean environment having a predetermined high cleanness. A move direction 1d of the cell aggregate from the cultivation plate supplying area 4 to the sticking area 6 via the transfer area 5 in the cell structure manufacturing system 1 is arranged along the access opening 11. The safety cabinet 3 is a space isolated from the external environment, so as to be separated from an environmental atmosphere, in order to prevent contamination of the internal closed space 1a. The safety cabinet 3 is a cabinet in which an environment of class II (JIS K3800:2009) or higher for the cabinet for biohazard countermeasures is guaranteed. The closed space 1a internally includes an access space 1b that can be accessed from the access opening 11. The access space 1b includes a pipette tip collecting room 5a that stores a removed thing from the transfer area 5 in a clean environment having a high cleanliness, and a collecting room 6a that stores a removed thing from the sticking area 6 in a clean environment having a high cleanliness. The closed space 1a including the pipette tip collecting room 5a and the collecting room 6a is the space where the environment of class II (JIS K3800:2009) or higher is guaranteed. The access space 1b and the pipette tip collecting room 5a communicate with each other via an opening 5b of a table surface 3a, and the access space 1b and the collecting room 6a communicate with each other via an opening 6b of the table surface 3a. The pipette tip collecting room 5a and the collecting room 6a are arranged in the direction that is perpendicular to the move direction 1d of the cell aggregate from the cultivation plate supplying area 4 to the sticking area 6 via the transfer area 5, and that is downward. Here, although the pipette tip collecting room 5a and the collecting room 6a for the removed thing from the sticking area 6 are divided, these may be integrated into one collecting room. It can be freely set according to the separation requirement for collected things.

Additionally, since a device constituting the cell structure manufacturing system 1 is placed on the table surface 3a, various holes are arranged. Therefore, an auxiliary space 1c can be arranged under the access space 1b. In this case, the auxiliary space 1c is also inside the closed space 1a. The access space 1b and the auxiliary space 1c are divided by the table surface 3a. Although it is not necessary to physically and strictly divide the respective spaces for the access space 1b and the auxiliary space 1c under the access space 1b, in order to maintain the environment of the access space 1b, it is preferable to make the pressure in the auxiliary space 1c lower than the pressure in the access space 1b, so as to prevent the flow of air from the auxiliary space 1c to the access space 1b.

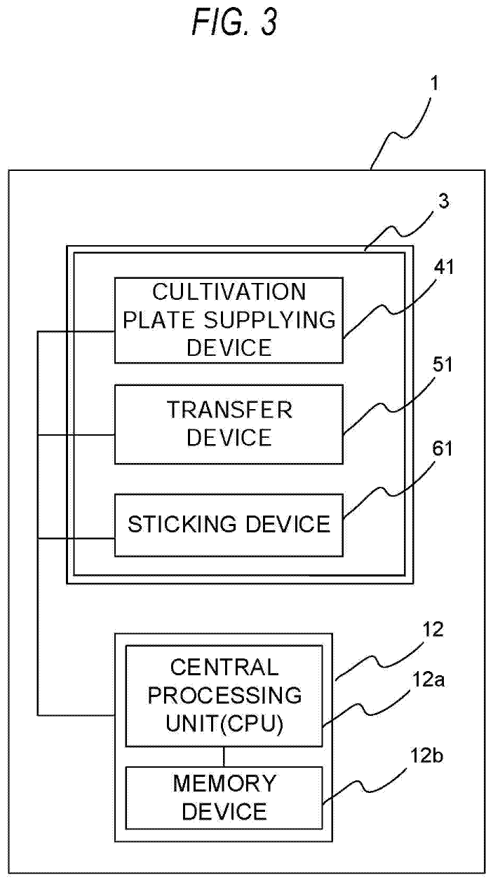

FIG. 3 is a configuration diagram of the cell structure manufacturing system 1. The cell structure manufacturing system 1 includes a cultivation plate supplying device 41, a transfer device 51, a sticking device 61, and a control device 12. Typically, the cultivation plate supplying device 41, the transfer device 51, and the sticking device 61 are arranged in the safety cabinet 3 at the positions corresponding to a cultivation plate supplying area 4, a transfer area 5, and a sticking area 6, respectively. Additionally, the control device 12 is arranged outside the safety cabinet 3. The control device 12 includes a central processing unit (CPU) 12a and a memory device 12b. The control device 12 is electrically connected to each of the cultivation plate supplying device 41, the transfer device 51, and the sticking device 61, and controls the operation of each of the devices.

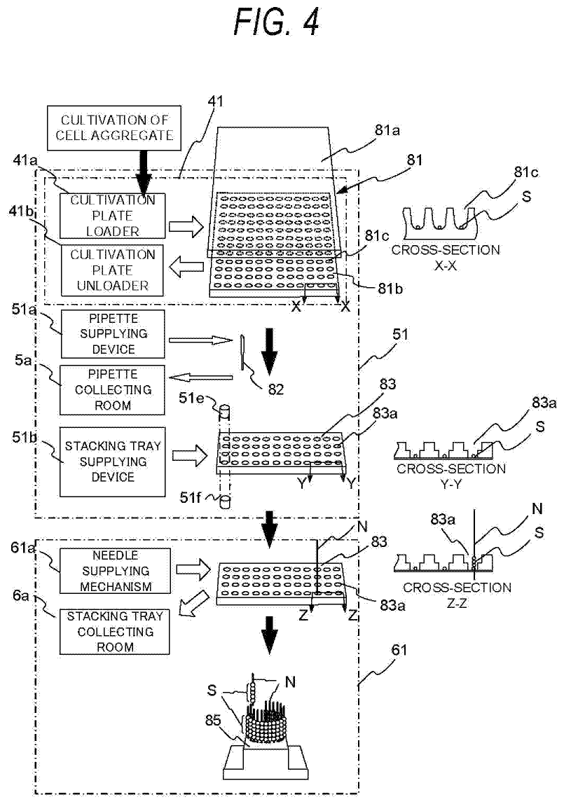

FIG. 4 shows a work process diagram of the cell structure manufacturing system 1. The cell aggregate S is cultivated and generated in the cultivation plate 81 outside the cell structure manufacturing system 1. The cultivation plate 81 is a rectangular standard cultivation plate, and has a shape that can cover a body 81b with a lid 81a. The body 81b has, for example, 16 rows in a long side direction, and four rows in a short side direction, i.e., a total of 64 wells 81c. A cross-section X-X of FIG. 4 shows a cross-section of the well 81c. The bottom of the well 81c does not have a function to allow penetration by a needle N, which is a needle-like body, and the cell aggregate S that has been cultivated and generated is supported by the bottom of the well 81c. The cultivation plate 81 is placed on a cultivation plate loader 41a of the cultivation plate supplying device 41 of the cell structure manufacturing system 1, in the state where the body 81b is covered by the lid 81a.

First, the cultivation plate supplying device 41 will be described. It is possible to place a plurality of cultivation plates 81 on the cultivation plate loader 41a at once, such that the cultivation plates 81 are stacked. When placing the cultivation plates 81 on the cultivation plate loader 41a of the cultivation plate supplying device 41, a user may perform the placement manually, or the placement may be performed automatically. Since it is necessary for the bottom of the well 81c to immerse the cell aggregate S in a culture solution for a predetermined period, the bottom of the well 81c has a nonporous structure capable of maintaining the culture solution. That is, since the bottom of the well 81c does not have a function to allow penetration by the needle N, in order to stick the cell aggregate S, it is necessary to move, to the bottom, a stacking tray 83 for sticking that has a function to allow penetration by the needle N. Therefore, the cultivation plate loader 41a automatically removes the placed cultivation plates 81 one by one, removes the lid 81a from the body 81b, and sends it to the transfer device 51 in the state where the wells 81c are exposed. Then, although the cell aggregates S in the wells 81c are moved to the exclusive stacking tray 83 for sticking of the cell aggregates S by the transfer device 51, the cultivation plate supplying device 41 and the transfer device 51 can be operated in parallel. That is, while sending out the cultivation plate 81 to the transfer device 51, the body 81b of the cultivation plate 81 after the cell aggregate S in all the wells 81c are moved to the stacking tray 83 by the transfer device 51 is returned to the cultivation plate supplying device 41 from the transfer device 51. The cultivation plate supplying device 41 puts the lid 81a on the body 81b of the empty cultivation plate 81 returned from the transfer device 51, and stores it in the cultivation plate unloader 41b. By repeating this, the empty cultivation plates 81 are stored in the cultivation plate unloader 41b.

Subsequently, the transfer device 51 will be described. The transfer device 51 includes a stacking tray supplying device 51b. Additionally, the transfer device 51 includes a plurality of suction pipes. A removable and disposable pipette tip 82 can be attached to each of the suction pipes. Each suction pipe can render the inside of the pipette tip 82 into a pressure-reduced state, and can cancel the pressure-reduced state, in the state where the pipette tip 82 is attached to the suction pipe. The transfer device 51 includes a pipette tip supplying device 51a, and the pipette tip supplying device 51a stores, for example, a plurality of pipette tips 82. The pipette tips 82 corresponding to the number of the suction pipes are pulled out from the pipette tips 82 stored in the pipette tip supplying device 51a, and are attached to a plurality of suction pipes. A plurality of stacking trays 83 can be stored in the stacking tray supplying device 51b, and are supplied from the stacking tray supplying device 51b according to the timing at which the cultivation plate 81 is supplied from the cultivation plate supplying device 41. As shown by a cross-section Y-Y of FIG. 4, the stacking tray 83 includes cell aggregate holding holes 83a, and a sheet of a porous material (for example, nonwoven fabric) that allows penetration by the needle N, and can support the cell aggregates S is attached to the bottom surfaces of the cell aggregate holding holes (hereinafter, the well holes) 83a. Typically, the stacking tray 83 has, for example, 16 rows in the long side direction, and four rows in the short side direction, i.e., a total of 64 well holes 83a. Therefore, it is ensured that the stacking tray 83 is supplied from the stacking tray supplying device 51b, so as to follow the supplying of one cultivation plate 81 from the cultivation plate supplying device 41 without delay.

The plurality of suction pipes can go back and forth between the top of the cultivation plate 81 carried from the cultivation plate supplying device 41, the top of the stacking tray 83, and the pipette tip supplying devices 51a. The number of the plurality of suction pipes can be determined in terms of the facility cost of the suction pipes. For example, it may be a common divisor of the number of the well holes 83a on a short side. In this case, in the stacking tray 83 where the 16 rows of well holes 83a are arranged in the short side direction, the number of the suction pipes is selected from 16, 8, 4, 2 or 1. According to the balance between the recovery in the case where an erroneously transferred cell aggregate S exists, and the throughput of manufacture, etc., it is possible to variously set the installation number of the suction pipes and the assembly of control sequence of transferring. First, the pipette tips 82 are supplied from the pipette tip supplying device 51a to the plurality of suction pipes, and the pipette tip 82 is attached to the tip of each of the plurality of suction pipes. For example, the pipette tip supplying device 51a is a commercially available pipette tip box where a predetermined number of pipette tips 82 are stored, the suction pipes are slid to be above the positions of the respectively corresponding pipette tips 82, and are moved down there, and the pipette tips 82 fit the tips of the suction pipes. It is possible to move up the suction pipes from there, and to attach the pipette tip 82 to the tip of each of the plurality of suction pipes.

After the pipette tip 82 is attached to the tip of each of the plurality of suction pipes, it is moved onto the cultivation plate 81, and sucks and stores the cell aggregate S in each well 81c of the cultivation plate 81 into the pipette tip 82 together with the culture solution. When the cell aggregates S are received into the plurality of suction pipes, the plurality of suction pipes are moved to be above the stacking tray 83, and discharge the cell aggregates S in the pipette tips 82 into the well holes 83a of the stacking tray 83. This is repeated, so that the cell aggregates S in all the wells 81c of the cultivation plate 81 are moved to all the well holes 83a of the stacking tray 83. As soon as moving of a predetermined number of sheets is completed, the plurality of suction pipes discard the pipette tips 82 attached to the tips, if necessary. The pipette tips 82 are discarded and collected into the pipette tip collecting room 5a through the opening 5b as the removed thing of the transfer area 5. For example, comb teeth-like claws that can hook the pipette tips 82 are arranged to the opening 5b, and the plurality of suction pipes for which the moving is completed are moved to the opening 5b, are moved down at the positions of the comb teeth-like claws, and operate so as to hook the pipette tips 82 of the respective plurality of suction pipes onto the comb teeth-like claws. When the plurality of suction pipes are moved up, the pipette tips 82 remain on the comb teeth-like claws, are detached from the suction pipes, fall from the opening 5b, and are collected into the pipette tip collecting room 5a.

Subsequently, the sticking device 61 will be described. The sticking device 61 sticks the cell aggregates S in the well holes 83a of the stacking tray 83 with the needles N. The sticking device 61 includes a needle supplying mechanism 61a, and sticks the cell aggregates S with the needles N supplied from the needle supplying mechanism 61a. As in a cross-section Z-Z, in the sticking device 61, the needle N is moved to be above the well hole 83a of the stacking tray 83, and is moved down from there so that the tip of the needle N sticks the cell aggregate S. Further, in the state where the needle N is moved down, since the tip of the needle N can penetrate the sheet of the porous material on the bottom of the well hole 83a, it is possible to obtain the state where the cell aggregate S is skewered by the needle N. When the cell aggregate S is penetrated by the needle N, the needle N is moved to the next well hole 83a, and sticks and penetrates the cell aggregate S in the well hole 83a. This is repeated until a predetermined number of cell aggregates S are penetrated by one needle N. The needle N with the predetermined number of skewered cell aggregates S is moved to be above an alignment base 85 made of a flexible form, and stabs the alignment base 85 with the needle N. The needle supplying mechanism 61a supplies the next needle N, and these are repeated, so that the needles N with the skewered cell aggregates S stab the alignment base 85 to be assembled in a predetermined shape on the alignment base 85. When all the cell aggregates S in the stacking tray 83 are stuck with the needles N, the emptied stacking tray 83 is discarded and collected into the collecting room 6a through the opening 6b as the removed thing of the sticking area 6.

The cell aggregate S in the well hole 83a is imaged by a luminescent device 51e and an imaging device 51f, and the image data is stored in the memory device 12b of the control device 12. The data is data of the center position of the cell aggregate S in the contour shape of the well hole 83a. The needle N reads the data from the memory device 12b as the correct position of the center position of the cell aggregate S in the well hole 83a, and drops the needle N to the center position of the cell aggregate S by the CPU. Typically, one of the luminescent device 51e and the imaging device 51f is arranged above the stacking tray 83, and the other is arranged below the stacking tray 83, and the inside of the well hole 83a is illuminated with the light emitted from the luminescent device 51e, while the transmitted light from the well hole 83a is received and imaged by the imaging device 51f. As in this embodiment, since it is efficient to perform the photography of the cell aggregate S in the well hole 83a at the same time in the process of transferring the cell aggregate S to the stacking tray 83, which is a process before sticking the cell aggregate S with the needle N, the photography of the cell aggregate S in the well hole 83a may be performed in the transfer area 5, while rendering the luminescent device 51e and the imaging device 51f as a part of the transfer devices 51. However, it is possible to perform in the sticking area 6, while rendering the luminescent device 51e and the imaging device 51f as a part of the sticking devices 61.

### Example 1

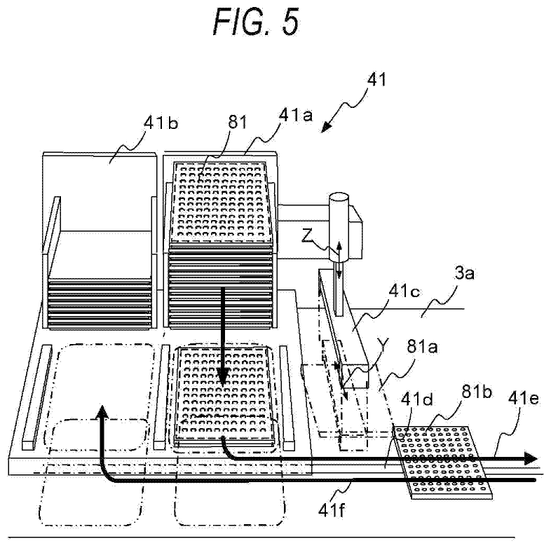

Subsequently, referring to FIG. 5 to FIG. 7, a description will be given of an example of the cell structure manufacturing system 1 that specifically achieves the aforementioned embodiment. Hereinafter, in addition to the matters already described in the embodiment, the characterizing portion in Example 1 will be additionally described. Also in Example 1, as shown in FIG. 1 to FIG. 3, the closed space 1a inside the cell structure manufacturing system 1 is the sealed space that can achieve the clean environment having the predetermined high cleanliness, and typically is the safety cabinet 3. The safety cabinet 3 is a so-called safety cabinet for biohazard countermeasures. The safety cabinet 3 is installed in a bioclean room maintained at the grade B, which is a workroom space in a cell processing center (CPC) that is a structure facility for performing, for example, cell processing, and the inside of the safety cabinet 3 is maintained at the grade A. The cell structure manufacturing system 1 includes the cultivation plate supplying device 41, the transfer device 51, the sticking device 61, and the control device 12. Among these, in the closed space 1a of the safety cabinet 3, the cultivation plate supplying device 41, the transfer device 51, and the sticking device 61 are arranged so as to correspond to the cultivation plate supplying area 4, the transfer area 5, and the sticking area 6, respectively. As for this part, it is as already described in the embodiment. In Example 1, the cultivation plate supplying area 4, the transfer area 5, and the sticking area 6 are arranged in a line along the access opening from the cultivation plate supplying area 4 to the sticking area 6 via the transfer area 5. The control device 12 can be made into various forms, such as arranging the central processing unit 12a inside a cabinet, and arranging a controller panel, etc. outside the cabinet.

First, referring to FIG. 5, the cultivation plate supplying device 41 in Example 1 will be described. A shelf-like cultivation plate loader 41a and a shelf-like cultivation plate unloader 41b are removably provided together in the cultivation plate supplying device 41. A plurality of cultivation plates 81 can be placed in the shelf of the cultivation plate loader 41a. For example, it is possible to place ten cultivation plates 81 in a stacked state. The cultivation plates 81 can be placed in the state where the cultivation plate loader 41a is removed from the cultivation plate supplying device 41, and the cultivation plate loader 41a on which the cultivation plates 81 are placed can be attached to the cultivation plate supplying device 41 as a unit. The cultivation plates 81 that are emptied by moving the cell aggregates S to the stacking tray 83 are stacked on top of each other in the cultivation plate unloader 41b, in the state where the cultivation plate unloader 41b is attached to the cultivation plate supplying device 41. When a predetermined number of emptied cultivation plates 81 are stacked on top of each other, the cultivation plate unloader 41b is removed, and the emptied cultivation plates 81 are collected. Since the cultivation plate loader 41a and the cultivation plate unloader 41b can be attached and detached in a unit manner, access from the outside can be cut off until all the works to the sticking process completely end, and the cleanness can be maintained in the closed space 1a in the safety cabinet 3 of the cell structure manufacturing system 1.

The cultivation plates 81 are automatically moved down one at a time from the shelf of the cultivation plate loader 41a in which a plurality of cultivation plates 81 are placed. The cultivation plate loader 41a moves down one of the cultivation plates 81 to be transferred onto the conveying device 41d. The conveying device 41d is a device that moves the cultivation plate 81 to the transfer area 5 on the table surface 3a. The conveying device 41d may be, for example, a device that can move back and forth between the cultivation plate supplying device 41 and the transfer device 51, or may be in the form where a table on which the cultivation plates 81 can be placed moves on a rail, or may be a moving device in the form where a caterpillar or an endless belt, etc. is rotated. The cultivation plate 81 placed on the conveying device 41d is conveyed toward a direction 41e of the transfer area 5. The conveying device 41d passes under a lid open/close arm 41c on the way. When the cultivation plate 81 passes under the lid open/close arm 41c, the lid open/close arm 41c holds and removes the lid 81a from the body 81b. The lid open/close arm 41c can be made into various forms, as long as the lid 81a of the cultivation plate 81 can be removed from the body 81b. For example, it can be a lid-removing gripper mechanism that grips and lifts the lid 81a by driving two air cylinders, i.e., driving of an air cylinder capable of moving in a first axial direction (holding direction A), so that one of the pairs of sides of a rectangular shape of the lid 81a can be held from a direction perpendicular to the pair of sides, and an air cylinder capable of moving the lid 81a in a second axial direction (up and down direction B), which is the vertical direction to the lid 81a. The body 81b of the cultivation plate 81 from which the lid 81a has been removed is conveyed to a predetermined position of the transfer area 5 by the conveying device 41d, and the cell aggregates S are transferred to the stacking tray 83. On the other hand, the empty cultivation plate 81 for which transferring of the cell aggregates S to the stacking tray 83 is completed is returned in the opposite direction 41f by the conveying device 41d, and is received in the cultivation plate unloader 41b.

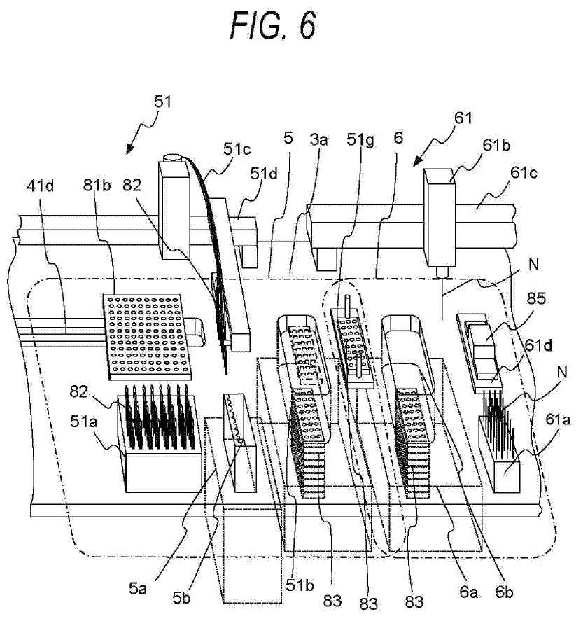

Subsequently, referring to FIG. 6, a description will be given of the transfer device 51 and the sticking device 61 in Example 1. The cultivation plate 81 is transported between the cultivation plate supplying area 4 and the transfer area 5. That is, the body 81b of the cultivation plate 81 placed and conveyed on the conveying device 41d stops at the predetermined position of the transfer area 5. The transfer device 51 includes the pipette tip supplying device 51a and the stacking tray supplying device 51b. For example, each of a plurality of pipette tips 82 is stored in the pipette tip supplying device 51a so as to extend in a substantially vertical direction. The transfer device 51 includes the suction pipes 51c, which are a predetermined number of injection devices determined as described in the embodiment. The pipette tip 82 can be attached to the tip of the suction pipe 51c so as to perform fluid communication. Each suction pipe 51c is connected to a decompression cylinder, and when the inside of the pipette tip 82 is decompressed by its pressure control, the pipette tip 82 sucks the cell aggregate S together with the culture solution from the tip, and when the decompression in the pipette tip 82 is canceled, the cell aggregate S can be discharged from the tip of the pipette tip 82. The transfer device 51 includes the stacking tray supplying device 51b, and can store a plurality of stacking trays 83 in advance. For example, the stacking tray supplying device 51b can be configured to be arranged below the table surface 3a, and to supply one of the stored stacking trays 83 so as to be moved up from the stacking tray supplying device 51b according to the timing at which the cultivation plate 81 is supplied to a predetermined position from the cultivation plate supplying device 41. The supplied stacking tray 83 is fixed onto a stacking tray fixing stage 51g. The suction pipes 51c are attached to a rail 51d, and are movable in two axial directions on a horizontal surface parallel to the table surface 3a. It is movable between the position at which the body 81b of the conveyed cultivation plate 81 is stopped, and the stacking tray fixing stage 51g.

First, the pipette tips 82 are supplied from the pipette tip supplying device 51a to the suction pipes 51c, and the pipette tip 82 is attached to the tip of each of the suction pipes 51c. Then, they are moved onto the conveyed cultivation plate 81, and suck and store the cell aggregates S in the respective wells 81c of the cultivation plate 81 into the pipette tips 82 together with the culture solution. When the cell aggregates S are received into the suction pipes 51c, the suction pipes 51c are moved onto the stacking tray 83 fixed on the stacking tray fixing stage 51g, and discharge the cell aggregates S in the pipette tips 82 into the well holes 83a of the stacking tray 83. By repeating this, the cell aggregates S in all the wells 81c of the cultivation plate 81 are moved to all the well holes 83a of the stacking tray 83. After the moving is completed, the pipette tips 82 attached to the tips of the suction pipes 51c are discarded and collected into the pipette tip collecting room 5a through the opening 5b drilled in the table surface 3a. A mechanism may be adopted in which the claws for hooking the pipette tips 82 are arranged in the opening 5b, and when the suction pipes 51c are moved up and down, the pipette tips 82 are hooked by these claws, and fall into the pipette tip collecting room 5a.

Subsequently, similarly referring to FIG. 6, the sticking device 61 will be described. The sticking device 61 includes the needle supplying mechanism 61a, which is a needle-like body supplying mechanism, a needle clamping mechanism 61b, which is a needle-like body clamping mechanism, and an alignment base fixing stage 61d. The needle supplying mechanism 61a can be made into various forms. For example, the needle supplying mechanism 61a can be made into the shape of a rack with a plurality of holes, where a plurality of needles N capable of sticking the cell aggregates S are stored in the respective holes so as to be aligned to extend in the vertical direction, so that the sharp sides of the plurality of needles N sticking the cell aggregates S become the lower sides. On the other hand, it is also possible to make the needle supplying mechanism 61a into the form of a mechanism in which the needles N are delivered one at a time to the needle clamping mechanism 61b. The needle clamping mechanism 61b can hold one needle N received from the needle supplying mechanism 61a. The alignment base 85, which is the flexible form made of silicon, etc., is fixed to the alignment base fixing stage 61d. The needle clamping mechanism 61b is attached to the rail 61c, is movable between the top of the stacking tray fixing stage 51g and the alignment base fixing stage 61d, and is configured to be able to move up and down. The needle clamping mechanism 61b is moved to the needle supplying mechanism 61a, holds one needle N, and is moved to be above the stacking tray fixing stage 51g. The control device 12 moves the needle N to be above the center of the cell aggregate S, based on the data of the position of the cell aggregate S stored in the memory device 12b in advance, and moves down the needle N toward the cell aggregate S in the well hole 83a of the stacking tray 83 to stick the cell aggregate S. When the needle N is moved down by a predetermined amount, and the tip of the needle N penetrates through the cell aggregate S by penetrating the sheet of the porous material on the bottom of the well hole 83a, the needle N is moved up, is moved to the next well hole 83a, and repeats the same operation. The needle N with a predetermined number of skewered cell aggregates S is moved onto the alignment base 85 on the alignment base fixing stage 61d, and the alignment base 85 is stabbed by the needle N. The needle supplying mechanism 61a supplies the next needle N, and these are repeated, so that the needles N with the skewered cell aggregates S stab the alignment base 85 to be assembled into a predetermined shape on the alignment base. This shape is controlled by the control device 12 to form the shape stored in the memory device 12b in advance. When all the cell aggregates S in the stacking tray 83 are stuck by the needles N, the emptied stacking tray 83 is discarded and collected into the collecting room 6a through the opening 6b of the table surface 3a.

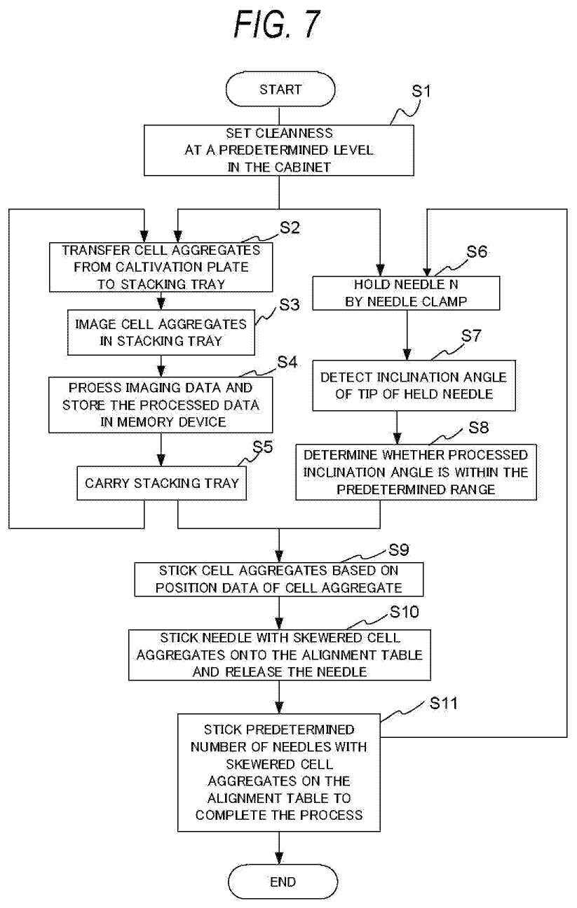

Subsequently, referring to FIG. 7, a description will be given of the flow of operations of the cell structure manufacturing system 1. FIG. 7 shows a flowchart of each process of the cell structure manufacturing system 1. First, as a preparatory step, a predetermined number of cultivation plates 81 storing the cell aggregates S are stored in the shelf of the cultivation plate loader 41a, and it is installed to the cultivation plate supplying device 41. Then, after confirming that the cleanness inside the safety cabinet 3 of the cell structure manufacturing system 1 reaches a stationary state, an automatic sequence is started by the control device 12 (S1). Subsequently, the cultivation plate 81 is conveyed by the cultivation plate supplying device 41, and movement to the well holes 83a of the stacking tray 83 is made by the transfer device 51 (S2). The cell aggregates S in the well holes 83a of the stacking tray 83 are imaged at the time of transfer to the stacking tray 83 (S3). The imaged data is analyzed by the central processing unit 12a of the control device 12, and is stored in the memory device 12b (S4).

On the other hand, on the sticking device 61 side, the needle clamping mechanism 61b grips a predetermined needle N kept in the needle supplying mechanism 61a (S6). Subsequently, the inclination angle with respect to the vertical direction of the needle N held by the needle clamping mechanism 61b is detected by a needle angle detector (S7). The control device 12 analyzes the detection result by the central processing unit 12a, and stores it in the memory device 12b. When the inclination angle of the needle N according to the analysis result is within a predetermined range, the central processing unit 12a proceeds to the next process, and when the inclination angle of the needle N is larger than the predetermined range, the central processing unit 12a controls to hold a new needle N, without performing sticking of the cell aggregate S by the needle N held by the needle clamping mechanism 61b (S8).

When the inclination angle of the needle N is within the predetermined range, the needle clamping mechanism 61b is moved to be above the center position of the cell aggregate S calculated according to the position and shape of the cell aggregate S stored in the memory device 12b of the control device 12, and the needle clamping mechanism 61b is moved down to stick the cell aggregate S. This process is repeated for a predetermined number of times (S9). The needle clamping mechanism 61b is moved down to the alignment base 85 by a predetermined amount to press and stick the needle N with skewered cell aggregates to the alignment base 85. When the needle clamping mechanism 61b is moved down by the predetermined amount and the needle N stands on its own on the alignment base 85, the needle clamping mechanism 61b releases the gripping of the needle N (S10). The needle clamping mechanism 61b returns to the process of gripping the predetermined needle N, and repeats the processes of S6 to S10 until a predetermined number of needles N are stuck and aligned on the alignment base 85 (S11). While the processes of S6 to S10 are repeated, the processes of S2 to S5 are performed in parallel in the transfer device 51.

### Example 2

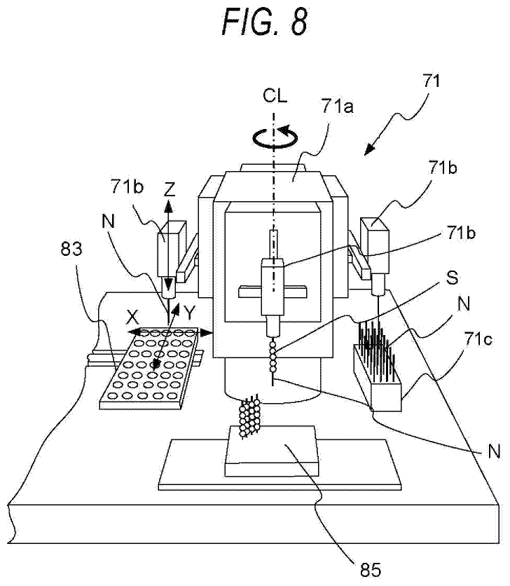

Subsequently, referring to FIG. 8, another form of a sticking device 71 will be described as Example 2. Except for the sticking device 71, it is the same as the cell structure manufacturing system 1 described in the embodiment and Example 1, and the same cultivation plate supplying device 41 and transfer device 51 as those in Example 1 are adopted. Here, only the sticking device 71, which is different from Example 1, will be described. In Example 2, the sticking device 71 includes a rotary unit 71a, a needle clamping mechanism 71b, a needle supplying mechanism 71c, and an alignment base 85. The rotary unit 71a is rotatable around a rotation axis extending in the vertical direction, and includes at least one attaching surface that is rotated by the rotation of the rotary unit 71a. The needle clamping mechanism 71b is attached to the attaching surface. Particularly, the rotary unit 71a has a polygonal shape having a plurality of attaching surfaces on the side surfaces, for example, a quadratic prism shape. That is, in the rotary unit 71a, four needle clamping mechanisms 71b are attached to the four side surfaces of a quadratic prism as “the attaching surfaces”. The rotary unit 71a is not limited to a quadratic prism, and may be a polygon such as a triangular prism, or may be a flat surface instead of a cubic shape. The needle clamping mechanisms 71b corresponding to the number of the side surfaces can be attached. In FIG. 8, an example is shown in which four needle clamping mechanisms 71b are attached to the rotary unit 71a. All of the four needle clamping mechanisms 71b have the same functions. The needle clamping mechanism 71b can move the needle N up and down in a vertical Z direction. The needle clamping mechanism 71b can grip and release the needle N in the state where the needle N extends in the vertical direction. Since the needle supplying mechanism 71c is the same as the needle supplying mechanism 61a in Example 1, a description is omitted. The sticking device 71 in Example 2 can perform parallel processing that performs in parallel each of the processes, i.e., the process of holding the needle N by the needle clamping mechanism 71b, and the process of sticking the cell aggregate S in the stacking tray 83, and the process of pressing and sticking the needle N with the skewered cell aggregates S to the alignment base 85, by rotating the rotary unit 71a around a central axis CL. At this time, three processes, i.e., the process of holding the needle N by the needle clamping mechanism 71b, the process of sticking the cell aggregate S in the stacking tray 83, and the process of pressing and sticking the needle N with skewered cell aggregates S on the alignment base 85, can be performed in divisions obtained by dividing the central angle of 360 degrees around the central axis CL with arbitrary ratios. Particularly, each of the divisions can be equally divided into 120 degrees. Additionally, a process of detecting the position of the tip of the needle N may be added between the process of holding the needle N, and the process of sticking the cell aggregate S in the stacking tray 83.

In the process of holding the needle N by the needle clamping mechanism 71b, the needle clamping mechanism 71b is located above the needle supplying mechanism 71c, and can move in three axial directions, i.e., a horizontal X direction, a horizontal Y direction perpendicular to the horizontal X direction, and the vertical Z direction. In the process of sticking the cell aggregate S in the stacking tray 83 by the needle clamping mechanism 71b, the needle clamping mechanism 71b is located above the stacking tray 83, and can move in the three axial directions, i.e., the horizontal X direction, the horizontal Y direction perpendicular to the horizontal X direction, and the vertical Z direction. In the process of pressing and sticking the needle N with skewered cell aggregates S to the alignment base 85 by the needle clamping mechanism 71b, the needle clamping mechanism 71b is located above the alignment base 85, and can move the needle N in the three axial directions, i.e., the horizontal X direction, the horizontal Y direction perpendicular to the horizontal X direction, and the vertical Z direction. That is, the needle supplying mechanism 71c, the stacking tray 83, and the alignment base 85 are arranged in order in the rotation direction of the rotary unit 71a. First, when one needle clamping mechanism 71b holds the needle N, the rotary unit 71a is rotated around the central axis CL and is moved to be above the stacking tray 83, and the needle clamping mechanism 71b moves down the needle N to stick the cell aggregate S in the stacking tray 83, and moves up the needle N. Here, simultaneously, another needle clamping mechanism 71b holds the needle N by receiving the needle N from the needle supplying mechanism 71c. Here, the rotary unit 71a is rotated around the central axis CL, and the needle clamping mechanism 71b holding the needle N with skewered cell aggregates S in the stacking tray 83 is moved to be above the alignment base 85, and presses and sticks the needle S with skewered cell aggregates S to the alignment base 85. At this time, the needle clamping mechanism 71b holding the needle N by receiving the needle N from the needle supplying mechanism 71c is moved to be above the stacking tray 83, moves down the needle N to stick the cell aggregate S in the stacking tray 83, and moves up the needle N. At this time, simultaneously, still another needle clamping mechanism 71b holds the needle N. The rotary unit 71a is rotated around the central axis CL, and performs this work to the cell aggregates S in all the well holes 83a. That is, by rotating the rotary unit 71a around the central axis CL at the termination stage of each process, transition can be made to the next process.

When adding the process of detecting the position of the tip of the needle N, four processes, i.e., the process of holding the needle N by the needle clamping mechanism 71b, the process of detecting the position of the tip of the needle N, the process of sticking the cell aggregate S in the stacking tray 83, and the process of pressing and sticking the needle N with skewered cell aggregates S to the alignment base 85, are performed in the respective divisions obtained by dividing the central angle of 360 degrees around the central axis CL with arbitrary ratios. Particularly, each of the divisions can be equally divided into 90 degrees. In the process of detecting the position of the tip of the needle N, the tip position in the surface defined by two directions, i.e., the horizontal X direction and the horizontal Y direction, is detected by a sensor. Transition is made to the process of sticking the cell aggregate S by moving the needle clamping mechanism 71b, so that this tip position is moved to be above the position of the cell aggregate S in the stacking tray 83 to be stuck, and here, the process of moving down the needle N to stick the cell aggregate S in the stacking tray 83, and moving up the needle N sticking the cell aggregate S by the needle clamping mechanism 71b is performed.

### REFERENCE SIGNS LIST

1 cell structure manufacturing system
3 safety cabinet
4 cultivation plate supplying area
5 transfer area
6 sticking area
41 cultivation plate supplying device
51 transfer device
61 sticking device
81 cultivation plate
82 pipette tip
83 stacking tray
85 alignment base
S cell aggregate
N needle

1. A cell structure manufacturing system for forming a cell structure by sticking a cell aggregate by a needle-like body, the cell structure manufacturing system comprising:
a cabinet including a closed space whose inside is maintained at a high cleanness, and an access opening enabling access from outside,
a cultivation plate supplying device configured to supply one cultivation plate of a plurality of cultivation plates to a conveying device in a cultivation plate supplying area, each cultivation plate having a plurality of cell aggregate receiving holes, the plurality of cell aggregate receiving holes storing externally generated cell aggregates,
a transfer device including a cell aggregate injection device configured to discharge and transfer, in a transfer area, the cell aggregates stored in the plurality of cell aggregate receiving holes of the one cultivation plate conveyed by the conveying device respectively to a plurality of cell aggregate holding holes of the stacking tray having the plurality of cell aggregate holding holes each allowing penetration of a bottom by the needle-like body, and
a sticking device including a needle-like body clamping mechanism configured to remove one needle-like body from a needle-like body supplying mechanism storing a plurality of needle-like bodies and perform gripping of the one needle-like body, the needle-like body clamping mechanism performing continuous sticking of each of the cell aggregates in the plurality of cell aggregate holding holes of the stacking tray by the one needle-like body in a sticking area, the needle-like body clamping mechanism pressing and sticking the needle-like body with the skewered cell aggregates to an alignment base to perform releasing of the gripping.

2. The cell structure manufacturing system according to claim 1,
wherein the conveying device transports the cultivation plate between the cultivation plate supplying area and the transfer area.

3. The cell stricture manufacturing system according to claim 2,
wherein the cultivation plate supplying area, the transfer area and the sticking area are arranged in a line along the access opening from the cultivation plate supply area to the sticking area via the transfer area.

4. The cell structure manufacturing system according to claim 3,
wherein the closed space is divided into divided spaces by a table surface, one of the divided spaces including the cultivation plate supplying area, the transfer area, and the sticking area, and another of the divided spaces including a collecting room configured to collects the stacking tray and the needle-like body.

5. The cell structure manufacturing system according to claim 4, wherein the sticking device is a rotatable polyhedron, and includes a rotary unit including a plurality of attaching surfaces, at least one attaching surface of the plurality of attaching surfaces includes a needle-like body clamping mechanism, and the rotary unit performs parallel processing of the gripping, the sticking and the releasing while being in rotation.

6. The cell structure manufacturing system according to claim 5, comprising a central axis that is a center of the rotation of the rotary unit, and in the sticking area, a process of holding the needle-like body by the needle-like body clamping mechanism, a process of sticking the cell aggregate in the stacking tray, and a process of pressing and sticking the needle-like body with the skewered cell aggregates to the alignment base are performed in divisions obtained by dividing a central angle about the central axis with arbitrary ratios.
